(19) United States
(12) Patent Application Publication
(10) Pub. No.: US 2025/0281289 A1
(43) Pub. Date: Sep. 11, 2025

(54) Percutaneous Delivery of Catheter

(71) Applicant: Cephea Valve Technologies, Inc., Abbott Park, IL (US)

(72) Inventors: Randolf Von Oepen, Aptos, CA (US); Jarred Hake, Fremont, CA (US); Austin Sherman, Sunnyvale, CA (US)

(73) Assignee: Cephea Valve Technologies, Inc., Abbott Park, IL (US)

(21) Appl. No.: 18/975,519

(22) Filed: Dec. 10, 2024

Related U.S. Application Data
(60) Provisional application No. 63/561,838, filed on Mar. 6, 2024.

(51) Int. Cl. A61F 2/24

(52) U.S. Cl. CPC A61F 2/2436

(57) ABSTRACT

A delivery system for delivering a prosthetic heart valve, the delivery system including: a handle assembly; a catheter assembly extending from the handle assembly, the catheter assembly having a first catheter and a second catheter positioned radially inside the first catheter; a connecting ring fixed to a distal end portion of the first catheter; and a valve retainer fixed to a distal end portion of the second catheter, the valve retainer configured to receive at least a portion of the prosthetic heart valve therein, wherein the catheter assembly has i) a locked state in which the valve retainer is axially locked relative to the connecting ring, and ii) an unlocked state in which the valve retainer is axially movable relative to the connecting ring, the handle assembly being configured to actuate the catheter assembly between the locked state and the unlocked state.

### CROSS-REFERENCE TO RELATED APPLICATIONS

The present application claims priority to U.S. Provisional Ser. No. 63/561,838, filed Mar. 6, 2024, the disclosure of which is hereby incorporated by reference in its entirety as if fully set forth herein.

### BACKGROUND

The present disclosure relates generally to devices, systems and methods for delivering an interventional device into a patient for implantation.

Intravascular medical procedures allow the performance of therapeutic treatments in a variety of locations within a patient's body while requiring only relatively small access incisions. An intravascular procedure may, for example, eliminate the need for open-heart surgery, reducing risks, costs, and time associated with an open-heart procedure. The intravascular procedure also enables faster recovery times with lower associated costs and risks of complications. An example of an intravascular procedure that significantly reduces procedure and recovery time and cost over conventional open surgery is a heart valve replacement or repair procedure in which an artificial valve or valve repair device is guided to the heart through the patient's vasculature. For example, a catheter is inserted into the patient's vasculature and directed to the inferior vena cava. The catheter is then urged through the inferior vena cava toward the heart by applying force longitudinally (e.g., pushed forward) to the catheter. Upon entering the heart from the inferior vena cava, the catheter enters the right atrium. The catheter may be guided across the atrial septum (e.g., via a guidewire that has already been passed through the atrial septum) into the left atrium. The distal end of the catheter may be deflected by one or more deflecting mechanisms in order to align the distal end of the catheter, as well as a medical device positioned therein, with the mitral valve. Catheter deflection can be achieved by tension cables, or other mechanisms positioned inside the catheter. Precise control of the distal end of the catheter allows for more reliable and faster positioning of a medical device and/or implant and other improvements in the procedures.

### BRIEF SUMMARY

One aspect of the present disclosure provides a delivery system for delivering a prosthetic heart valve, the delivery system comprising: a handle assembly; a catheter assembly extending from the handle assembly, the catheter assembly having a first catheter and a second catheter positioned radially inside the first catheter; a connecting ring fixed to a distal end portion of the first catheter; and a valve retainer fixed to a distal end portion of the second catheter, the valve retainer configured to receive at least a portion of the prosthetic heart valve therein, wherein the catheter assembly has i) a locked state in which the valve retainer is axially locked relative to the connecting ring, and ii) an unlocked state in which the valve retainer is axially movable relative to the connecting ring, the handle assembly being configured to actuate the catheter assembly between the locked state and the unlocked state.

In one example, the first catheter has a coiled or braided section positioned proximal to the connecting ring.

In one example, the second catheter has a greater resistance to longitudinal compression compared to the first catheter.

In one example, the first catheter is an outer sheath of the catheter assembly; and the second catheter is a steering catheter or an extension catheter.

In one example, the first catheter is an outer sheath of the catheter assembly; and the second catheter a steering catheter or an intermediate catheter.

In one example, the connecting ring defines a slot configured to receive a tab of the valve retainer.

In one example, the valve retainer defines a slot configured to receive a tab of the connecting ring.

In one example, the handle assembly includes a locking assembly such that rotation of the locking assembly relative to other components of the handle assembly actuates the catheter assembly between the locked state and the unlocked state.

In one example, the catheter assembly includes a valve cover fixed to the connecting ring, the valve cover configured to maintain at least part of the prosthetic heart valve in a collapsed condition.

In one example, in the locked state of the catheter assembly, the valve cover is axially locked relative to the valve retainer, thereby preventing premature expression of the prosthetic heart valve from the valve cover.

In one example, in the unlocked state of the catheter assembly, the valve cover is configured to retract proximally along with the first catheter relative to the second catheter and relative to the valve retainer, thereby allowing the for expression of the prosthetic heart valve from the valve cover.

In one example, the valve cover is threadedly coupled to the connecting ring.

In one example, the wherein the handle assembly includes (i) a valve cover retraction knob and (ii) a locking assembly, such that rotation of the valve cover retraction knob in the unlocked state of the catheter assembly proximally withdraws the valve cover relative to the valve retainer, and rotation of the locking assembly relative to other components of the handle assembly actuates the catheter assembly between the locked state and the unlocked state, the valve cover retraction knob being positioned distal to the locking assembly.

In one example, the valve retainer is threadedly coupled to the second catheter.

Another aspect of the disclosure provides a method of delivering a prosthetic heart valve, comprising: advancing a catheter assembly through a vasculature while the catheter assembly is in a locked state in which a first catheter is axially locked relative to a second catheter nested within the first catheter, and while the prosthetic heart valve is maintained in a collapsed condition within a valve cover coupled to a distal end of the first catheter; actuating the catheter assembly to an unlocked state, thereby allowing for axial movement of the first catheter relative to the second catheter; and retracting the valve cover and the first catheter relative to the second catheter to deploy the prosthetic heart valve into a native mitral valve annulus.

In one example, actuating the catheter assembly to the unlocked state is performed when a distal end of the catheter assembly is positioned within or adjacent to an opening of an inferior vena cava leading to a right atrium.

In one example, actuating the catheter assembly to the unlocked state includes rotating a locking assembly of a handle assembly, the catheter assembly extending from the handle assembly.

In one example, the catheter assembly includes a connecting ring that couples the valve cover to the distal end of the first catheter, and a valve retainer coupled to a distal end of the second catheter.

In one example, actuating the catheter assembly to the unlocked state includes rotating the first catheter relative to the second catheter such that the valve retainer rotates relative to the connecting ring.

### BRIEF DESCRIPTION OF THE DRAWINGS

A more complete appreciation of the subject matter of the present disclosure and the various advantages thereof can be realized by reference to the following detailed description in which reference is made to the accompanying drawings in which:

FIG. 1 is a perspective view of a delivery system configured for delivering, positioning and deploying a prosthetic heart valve, including a handle assembly and a catheter assembly;

FIG. 2 is an exploded view of a portion of the handle assembly of the delivery system of FIG. 1, including valve cover retraction knob and cone assembly;

FIG. 3 is a side view of a portion of the catheter assembly of the delivery system of FIG. 1, including a catheter shaft, coiled and/or braided section, valve cover, and nosecone;

FIG. 4A is a transverse cross-sectional view of the catheter shaft of FIG. 3, showing various components thereof;

FIGS. 4B-4C are transverse cross-sectional views of a catheter assembly, operable with the delivery system of FIG. 1, according to another aspect of the disclosure;

FIG. 5A is a longitudinal cross-section of the catheter assembly of FIG. 3, showing the nested arrangement of various components thereof;

FIG. 5B is a longitudinal cross-section of a catheter assembly, operable with the delivery system of FIG. 1 and according to another aspect of the disclosure, showing the nested arrangement of various components thereof;

FIG. 6 is a side view of two catheter assemblies, one catheter assembly in an uncompressed condition and another catheter assembly in a longitudinally compressed condition;

FIG. 7A is a perspective view of a connecting ring according to one or more aspects of the disclosure;

FIG. 7B is a perspective view of a valve retainer according to one or more aspects of the disclosure;

FIG. 7C is a longitudinal cross-section of the connecting ring of FIG. 7A coupled with the valve retainer of FIG. 7B;

FIG. 8A is a perspective view of a connecting ring according to one or more aspects of the disclosure;

FIG. 8B is a perspective view of a valve retainer according to one or more aspects of the disclosure;

FIGS. 8C-D are transverse cross-sectional views of the connecting ring of FIG. 8A coupled with the valve retainer of FIG. 8B;

FIG. 9A is a perspective view of a connecting ring according to one or more aspects of the disclosure;

FIG. 9B is a front view of the connecting ring of FIG. 9A according to one or more aspects of the disclosure;

FIG. 9C is a rear view of the connecting ring of FIG. 9A according to one or more aspects of the disclosure;

FIG. 9D is a perspective view of a valve retainer according to one or more aspects of the disclosure;

FIG. 9E is a front view of the valve retainer of FIG. 9D according to one or more aspects of the disclosure;

FIG. 9F is a rear view of the valve retainer of FIG. 9D according to one or more aspects of the disclosure;

FIG. 9G is a perspective view of the connecting ring of FIGS. 9A-C coupled with the valve retainer of FIGS. 9D-F according to one or more aspects of the disclosure;

FIG. 9H is a transverse cross-sectional view of the connecting ring of FIGS. 9A-C coupled with the valve retainer of FIGS. 9D-F according to one or more aspects of the disclosure;

FIG. 9I is a longitudinal cross-sectional view of the connecting ring of FIGS. 9A-C coupled with the valve retainer of FIGS. 9D-F according to one or more aspects of the disclosure;

FIG. 10A is a perspective view of a cone assembly operable with the delivery system of FIG. 1;

FIGS. 10B-C are side views of the handle assembly of FIG. 10A in unlocked and locked states, respectively;

FIG. 11 is a schematic cutaway view of the heart, showing an exemplary approach for delivering a prosthetic heart valve to a mitral valve annulus; and

FIG. 12 is a flow chart depicting example steps of a method of operating a handle assembly during delivery of the prosthetic heart valve to the mitral valve annulus.

### DETAILED DESCRIPTION

As used herein, the term “inflow end,” when used in connection with a prosthetic heart valve, refers to the end of the heart valve through which blood enters when the heart valve is functioning as intended, whereas the term “outflow end,” when used in connection with a prosthetic heart valve, refers to the end of the heart valve through which blood exits when the heart valve is functioning as intended. For a prosthetic mitral valve, the inflow end is closest to the left atrium when the heart valve is implanted in a patient, and the outflow end is closest to the left ventricle when the heart valve is implanted in a patient. Further, when used herein in connection with a delivery device, the terms “proximal” and “distal” are to be taken as relative to a user operating the device in an intended manner. “Proximal” is to be understood as relatively close to the user and “distal” is to be understood as relatively farther away from the user. Also as used herein, the terms “substantially,” “generally,” and “about” are intended to mean that slight deviations from absolute are included within the scope of the term so modified.

Native mitral and tricuspid valves, especially the native tricuspid valve, are typically larger in diameter than a native aortic valve. Thus, collapsible and expandable prosthetic mitral and tricuspid valves are typically much larger in diameter than collapsible and expandable prosthetic aortic and pulmonary valves. As a result, transcatheter delivery devices for prosthetic mitral and tricuspid valves are typically larger in size than transcatheter delivery devices for prosthetic aortic and pulmonary valves. It is generally desirable for transcatheter delivery devices to be as small as possible while still being capable of retaining the prosthetic heart valve therein and otherwise functioning as intended. Some transcatheter prosthetic heart valve delivery devices are used with separate introducers that are first inserted into the relevant vasculature (e.g., the femoral vein), and only after the introducers are in place, the delivery device is inserted into the vasculature through the introducer. In some instances, the introducers are necessary or at least important because they may prevent bleeding during the delivery procedure since the introducer typically has a hemostatic valve at its proximal end and provides a sealing function when no device is inserted into the delivery device. The hemostatic valve can also provide a sealing function around devices inserted into the vasculature during the procedure, such as guide wires, catheters, device(s) to cross the native septum, etc. The introducer is stagnant during the procedure, relative to the vasculature, such that the vasculature may be shielded against motion needed during the procedure (e.g., advancing, retraction, and/or rotation of a catheter). The introducer can allow for a catheter exchange. Further, the introducer may allow the delivery device to avoid direct contact with the patient's vasculature for at least some length of the delivery route. As described in greater detail below, one potential benefit of using an introducer to avoid direct contact between the delivery device and the patient's vasculature is that friction between the components may cause negative effects on the delivery device itself, for example causing longitudinal compression that might result in premature deployment of a prosthetic heart valve maintained therein. However, one negative effect of using an introducer is that, because the delivery device must pass through the introducer, the total size of the delivery device and introducer is larger than if the delivery device were able to be used without the introducer. Also, in some examples, a cut-down of the blood vessel will need to be performed to accommodate the overall larger diameter of the introducer. Thus, it may be desirable to be able to eliminate the need for a separate introducer and directly insert the delivery device into the vasculature. This may result in either (i) a reduction of the total size of the system passing into the patient's vasculature or (ii) the ability to increase the size of the delivery system (which may be beneficial) while maintaining the same overall diameter of the system because there is a concomitant reduction in size in view of the omission of the introducer. However, in order to eliminate the use of the introducer, the delivery device may need to be modified so that the typical benefit(s) of using the introducer are no longer needed. The disclosure provided herein may, in some instances, address one or more of these issues.

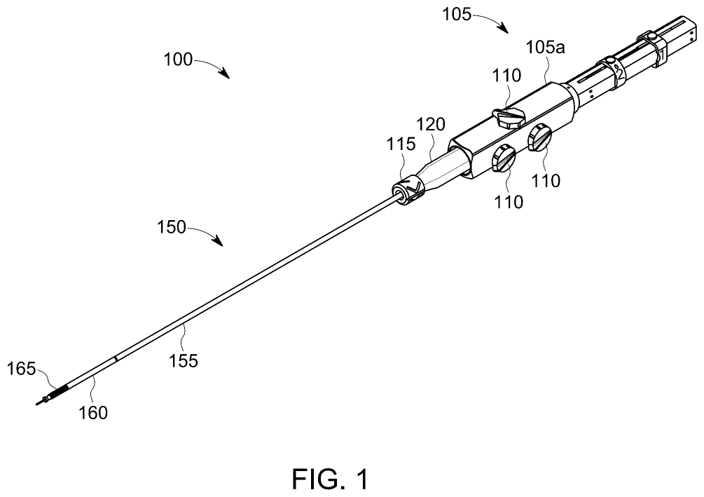

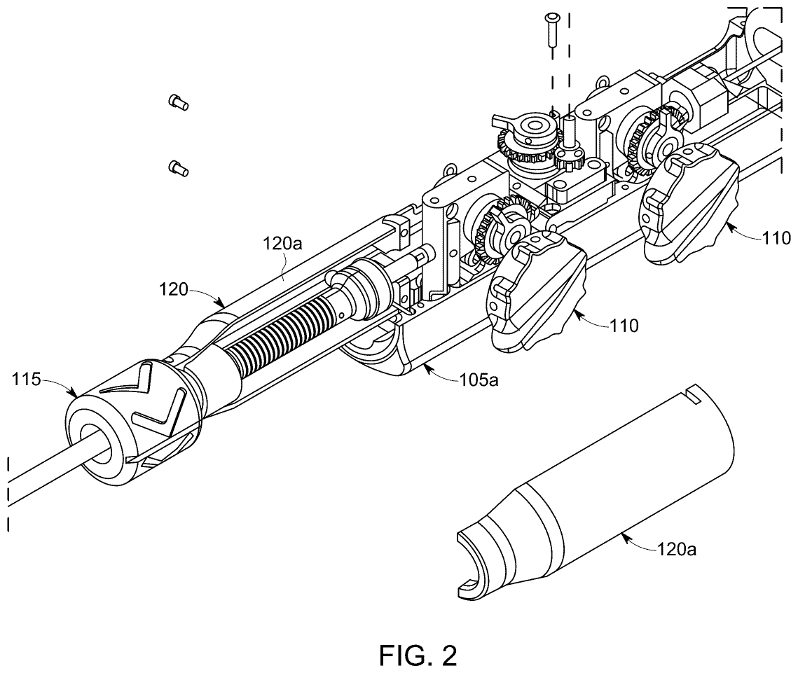

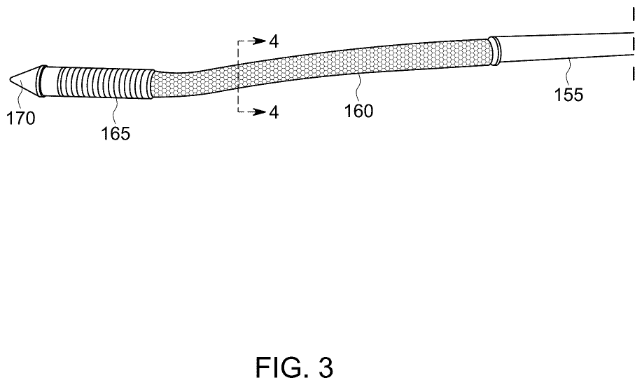

FIG. 1 is a perspective view of a delivery system 100 configured for delivering, positioning and deploying a prosthetic heart valve, including a handle assembly 105 and a catheter assembly 150. FIG. 2 is an exploded view of a portion of the handle assembly 105 of the delivery system 100 of FIG. 1, including valve cover retraction knob 115 and cone assembly 120 (which may also be referred to as a locking assembly herein). FIG. 3 is a side view of a distal end portion of the catheter assembly 150 of the delivery system 100 of FIG. 1, including a catheter shaft 155, coiled and/or braided section 160, valve cover 165 (which may also be referred to as a valve capsule), and an atraumatic distal tip or nosecone 170.

Delivery system 100 generally includes a handle assembly 105 and a catheter assembly 150. Catheter assembly 150 extends from a proximal end coupled to handle assembly 105 to an atraumatic tip or nosecone (not shown in FIG. 1) at a distal end and includes a plurality of catheter and/or hypotube components, at least some of which are longitudinally slidable relative to one another and provide different functionality during operation of delivery system 100 to enable effective delivery and deployment of a prosthetic heart valve, such as a prosthetic mitral valve. While the catheter assembly 150 is depicted as having a length relative to handle assembly 105, it should be understood that the catheter assembly 150 can have any suitable length and, in some examples, has a much greater length than the handle assembly 105.

Attached to a housing 105a of the handle assembly 105 can be various control mechanisms for controlling one or more aspects of the delivery. For example, attached to the housing 105a of the handle assembly 105 can be one or more knobs 110 for controlling steering of the of the catheter assembly 150. Additional details regarding steering operations that may be utilized in conjunction with the components and features described herein are described in U.S. Patent Application Publication No. 2023/0364387, filed Apr. 19, 2023, entitled “Advanced 3-Way Steering,” the disclosure of which is hereby incorporated by reference herein. The handle assembly 105 can also include a valve cover retraction knob 115 and a locking or cone assembly 120. The valve cover retraction knob 115 can be rotated to allow for retraction of valve cover 165 during delivery and/or expression of a prosthetic heart valve. In some examples, rotating valve cover retraction knob 115 results in proximal translation of a catheter member that is attached to the valve cover 165, allowing for the valve cover 165 to uncover a self-expandable prosthetic heart valve to allow the prosthetic heart valve to self-expand. The cone assembly 120 can be rotated relative to housing 105a, as is explained in greater detail below, to lock or unlock an outer catheter relative to an inner catheter.

The catheter assembly 150 can include a catheter shaft 155, a coiled and/or braided section 160, a valve cover 165, and a nosecone (not shown in FIG. 1), as will be described in greater detail below. It should also be understood that catheter assembly 150 may include a number of additional catheter members nested within catheter shaft 155, as described in greater detail below.

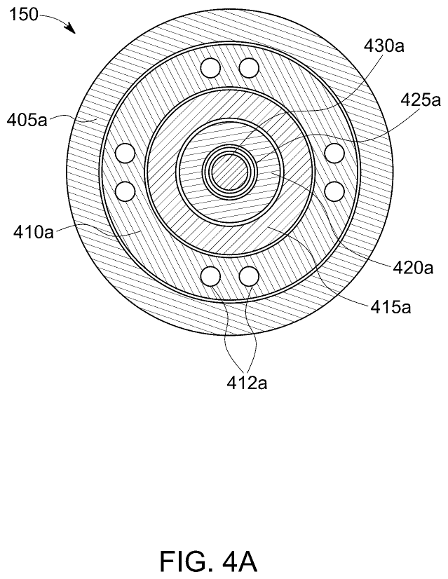

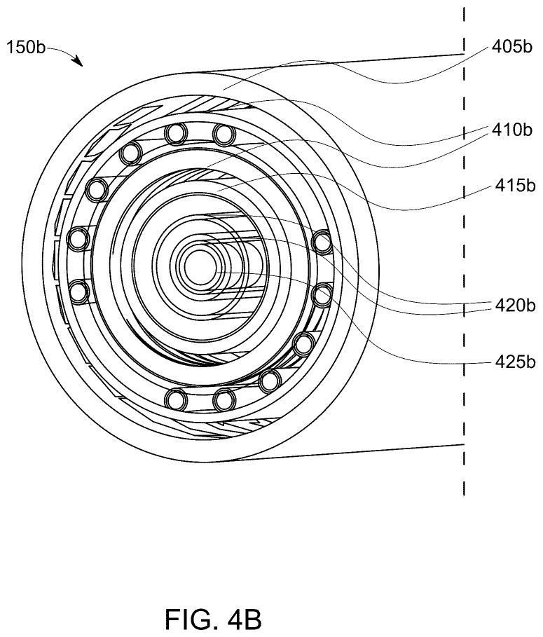

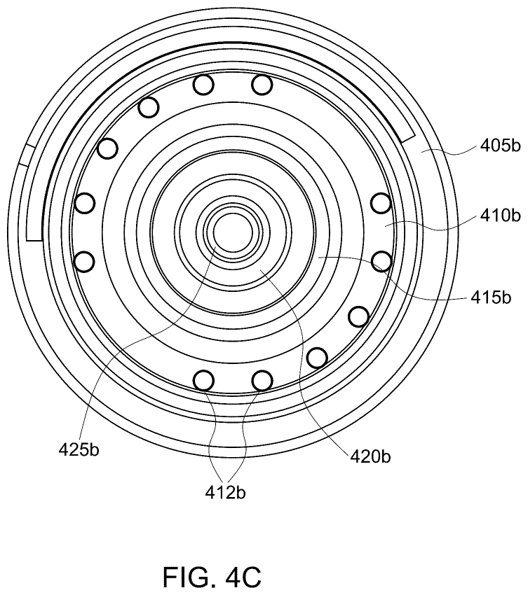

FIG. 4A is a transverse cross-sectional view of the catheter shaft 155 of FIG. 3, showing various components thereof, including addition catheter components nested therein.

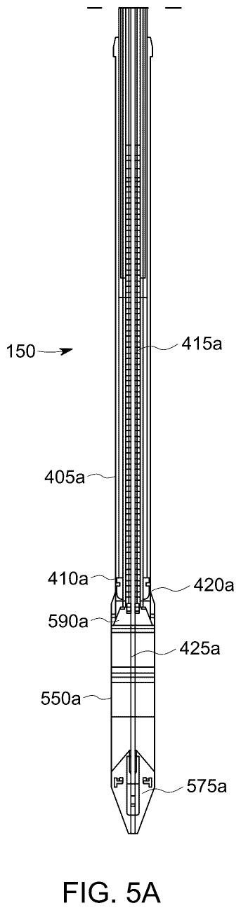

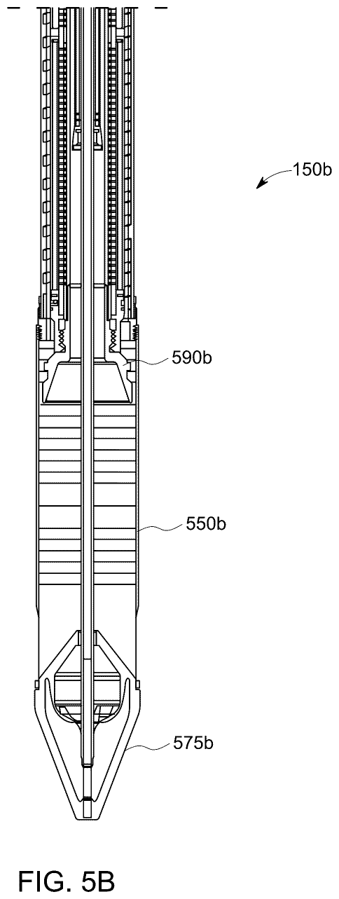

As shown in FIG. 4A, which is a transverse cross-sectional view of catheter assembly 150 taken along line 4-4 of FIG. 3, these components may include an outer sheath 405a, a steering catheter 410a, an extension catheter 415a, a suture catheter 420a, and a nosecone catheter 425a, all arranged in a concentric nested relationship. The arrangement of these components, as well as valve cover 550a, nosecone 575a, and valve retainer 590a (also referred to as “can”) is shown in the longitudinal cross-section of catheter assembly 150 shown in FIG. 5A. During delivery, the proximal petals of the compressed prosthetic valve can be nested inside of the valve retainer when the prosthetic valve is held back by the suture catheter, which is under tension. In one example, valve retainer 590a may be attached directly to a tip ring of the steering catheter 410a. As illustrated in the figures, nosecone catheter 425a has a lumen sized to receive a guidewire 430a therein. Each of these components is described in detail below. It should be understood that the valve cover 165 of FIG. 1 and FIG. 3 may be substantially the same as or identical to the other valve covers described herein. Similarly, it should be understood that the atraumatic distal tip or nosecone 170 of FIG. 3 may be substantially the same as or identical to the other atraumatic distal tips or nosecones described herein. In some examples, the suture catheter 420a may have a plurality of sutures or other wire loops attached to a distal end thereof, the suture loops or wire loops configured to selectively maintain a connection between the prosthetic heart valve and the delivery device.

To selectively control the curvature of a distal section of steering catheter 410a, the steering catheter 410a may be provided with a plurality of tension cables (not shown). The tension cables may travel from handle assembly 105 (e.g., one or more of the knobs 110) through a plurality of lumens 412a (which in some embodiments may be polymer tubes, such as Nylon, Pebax, Polyimide, or polytetrafluoroethylene or “PTFE” tubes, or any other type of polymer) to the distal end of steering catheter 410a. In one embodiment, steering catheter 410a may include four lumens 412a equally spaced at 90° intervals around the circumference of steering catheter 410a. In the embodiment of FIG. 4A, steering catheter 410a may include four pairs of lumens 412a (a total of eight lumens 412a), with the pairs of lumens equally spaced at 90° intervals around the circumference of steering catheter 410a. Any number of these lumens 412a (or pairs of lumens) may be provided depending on the various directions of deflection that may be desired. For example, six pairs of lumens 412a may be provided, with each pair of lumens 412a housing one steering cable of a pair of steering cables coupled to a corresponding steering knob 110 (e.g., two steering cables attached to each steering knob 110, with each steering cable extending through one pair of lumens 412a, for a total of twelve lumens 412a arranged in six pairs).

FIGS. 4B-4C are transverse cross-sectional views of a catheter assembly 150b, operable with the delivery system of FIG. 1 (e.g., in place of catheter assembly 150), according to another aspect of the disclosure.

In this example, catheter assembly 150b omits an extension catheter (e.g., extension catheter 415a described above) and includes an outer catheter 405b, a steering catheter 410b, an intermediate catheter 415b, a suture catheter 420b, and a nosecone catheter 425b. Also in this example, the steering catheter 410b includes six pairs (a total of 12) of lumens 412b, which again may be formed with polymer tubes. In this example, the extension catheter is omitted and an intermediate catheter 415b is implemented in its place. Implementation of the intermediate catheter 415b can advantageously reduce an overall diameter (e.g., French size) of the catheter assembly 150. A distal portion of the intermediate catheter 415b can be fixed (e.g., by a threaded connection) to a distal tip of the steering catheter 410b. The distal portion of the intermediate catheter 415b can have a portion of greater diameter, while the proximal portion can be adjacent to the suture catheter 420b to allow for smooth advancement/retraction of the suture catheter 420b during deployment.

The arrangement of these components, as well as valve cover 550b, nosecone 575b, and valve retainer 590b is shown in the longitudinal cross-section of catheter assembly 150b shown in FIG. 5B.

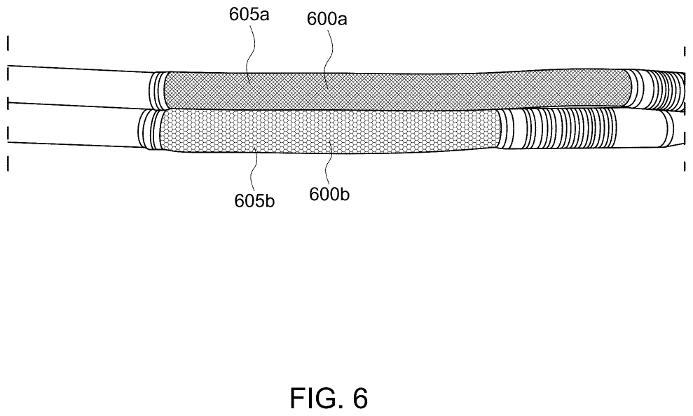

FIG. 6 is a side view of two catheter assemblies 600a, 600b, one catheter assembly 600a in an uncompressed condition and another catheter assembly 600b in a longitudinally compressed condition. As shown in FIG. 6, the coil/braid 605a, 605b (which may be substantially similar to coiled and/or braided section 160) may have very little resistance against compression. When the force to insert the catheter is larger than the release force of the valve, the coil/braid 605a, 605b may be longitudinally compressed and the prosthetic heart valve maintained in the valve cover (e.g., valve cover 165) may be prematurely released during delivery. As noted above, this potential problem may be largely or entirely avoided by the use of a separate introducer which is first inserted into the patients' vasculature, which may result in the frictional forces between the delivery device and the introducer during insertion to be lower than the release forces of the prosthetic valve, thereby allowing for safe delivery and/or deployment of the valve. However, in the absence of such an introducer, friction between the delivery device and the vasculature may be greatly increased, potentially resulting in the longitudinal compression shown in FIG. 6, and thus potentially resulting in premature deployment of the prosthetic heart valve from the capsule or valve cover 165.

The coiled and/or braided section 160 of the catheter assembly 150 advantageously allows for the application of high forces to unsheathe the prosthetic heart valve during delivery. In some examples, the unsheathe forces can be 50 pounds or more especially if the catheter is deflected in several planes at its distal end. When pull force to unsheathe the valve is applied, the braid will lengthen and lock down on the coil. This prevents ovalization of the catheter assembly even if tight bends are applied and can therefore move freely on the steering catheter.

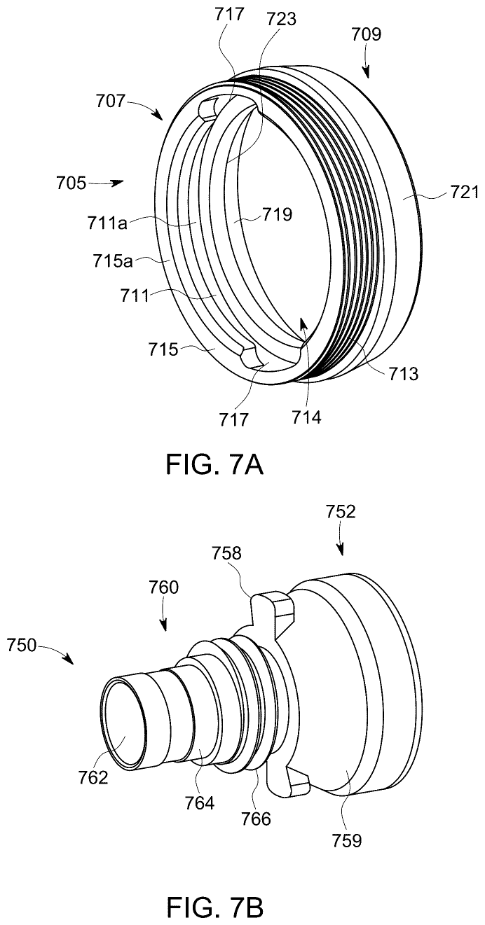

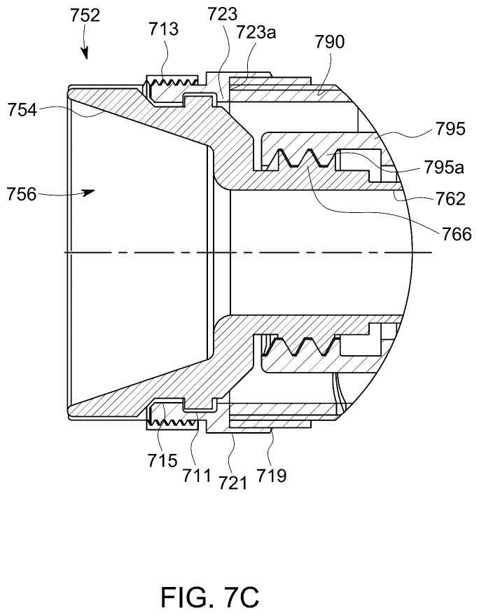

FIG. 7A is a perspective view of a connecting ring 705 according to one or more aspects of the disclosure. FIG. 7B is a perspective view of a valve retainer 750 according to one or more aspects of the disclosure. FIG. 7C is a longitudinal cross-section of the connecting ring 705 of FIG. 7A coupled with the valve retainer 750 of FIG. 7B.

As shown in FIG. 7A, the connecting ring 705 can be substantially cylindrical in shape and can have a distal portion 707 and a proximal portion 709. By virtue of the substantially cylindrical shape, the distal portion 707 can define an internal surface 711 and an external surface 713, with the external surface 713 having an external threading configured to engage with and couple to a valve cover (e.g., valve cover 550a or 550b). In other examples, the external surface may be unthreaded and may couple with a valve cover directly by laser welding.

The distal portion 707 of the connecting ring 705 can have a lip 715. The lip 715 can extend radially inward relative to the internal surface 711. The lip 715 can have a substantially planar face 715a that can be distal-facing and positioned at a distal end of the distal portion 707. In this regard, the lip has an internal diameter that defines an opening 714 through which a portion of the valve retainer 750 may be received.

The lip 715 can define one or more cutouts 717. In this regard, the cutouts 717 can be defined as cutout portions of the lip face 715a resulting in the opening 714 having an increased diameter at the cutouts as compared to at the remainder of the lip face 715a. The cutouts 717 can have any shape and the edges of the lip face 715a that define cutouts 717 can be any combination of linear, curved, or curvilinear. In the example of FIG. 7A, a pair of cutouts 717 are depicted as being approximately 180 degrees from one another, while in other examples any number of cutouts 717 in any angular orientations can be implemented. As will be explained in greater detail below, the shape of the cutouts 717, and the number and position of cutouts 717 are selected to allow for the connecting ring 705 to reversibly or selectively lock with valve retainer 750.

The proximal portion 709 can also define an internal surface 719 and an external surface 721. In one example, the internal surface 719 is unthreaded and the external surface 721 is unthreaded. As shown in FIG. 7C, the internal surface 719 can define a single internal diameter and the external surface 721 can define a single outer diameter. In one example, the internal diameter of the internal surface 719 is greater than one or all of the internal diameters defined by internal surface 711, lip 715, or external threading 713 of the distal portion 707.

The proximal portion 709 can define a lip 723. The lip 723 can have a substantially planar face 723a (labeled in FIG. 7C) that can be proximal-facing and positioned at a distal end of the proximal portion 709. A channel 711a is defined between lip 723 and lip 715.

The valve retainer 750 can have a distal portion 752 and a proximal portion 760. The distal portion 752 have an internal surface 754 (shown in FIG. 7C) defining at least one internal diameter. In one example, the internal surface defines an opening 756 (also referred to as a “valve seat”) configured to receive the tips of proximal petals of an atrial disk of a prosthetic valve in a collapsed state. The opening 756 can be generally cone-shaped or frustoconical, having an internal diameter that increases in the distal direction to correspond to a shape of compressed petals of the atrial disk of the prosthetic valve. In one example, the opening 756 can be the shape of a truncated cone, with the truncation being at a proximal end of the distal portion 752. The valve retainer 750 may have a general function that is similar or identical to that of valve retainers 590a, 590b, which is to receive an end (e.g., an atrial or inflow end) of a prosthetic heart valve within the opening 756, such that the end of the prosthetic heart valve is maintained in a collapsed condition by the internal surface 754, while the remainder of the prosthetic valve is maintained in the collapsed condition by the valve cover (e.g., valve cover 550a or 550b).

The distal portion 752 can define one or more tabs 758 that extend radially outward from an outer surface 759 of the distal portion 752. The tabs 758 can have any shape that is complementary to the shape of cutouts 717 and the edges that define tabs 758 can be any combination of linear, curved, or curvilinear. In the example of FIG. 7B, a pair of tabs 758 are depicted as being approximately 180 degrees from one another, while in other examples any number of tabs 758 in any angular orientations can be implemented in a fashion that is complementary to the number and orientation of cutouts 717. As will be explained in greater detail below, the number and position of tabs 758 are selected to allow for the connecting ring 705 to reversibly or selectively lock with valve retainer 750.

The proximal portion 760 can define an internal surface 762 and an external surface 764. At least a portion of the external surface 764 can be a threaded region 766.

As shown in FIG. 7C, the connecting ring 705 can engage and lock with the valve retainer 750. To do so, the distal portion 707 of connecting ring 705 is positioned to face the proximal portion 760 of valve retainer 750. The tabs 758 of the valve retainer 750 are first aligned with the cutouts 717 of the connecting ring 705 and then either or both of the valve retainer 750 and/or connecting ring 705 are advanced such that the tabs 758 pass through cutouts 717 and into channel 711a. In this position, the valve retainer 750 and/or connecting ring 705 are in the unlocked position, as the tabs 758 are free to move axially in a distal direction relative to the cutouts 717. Before rotation of either or both of the valve retainer 750 and/or connecting ring 705 from the unlocked position to a locked position, the tabs 758 are free to move axially in a distal direction relative to the cutouts 717 and thus the valve retainer 750 is free to move axially in a distal direction relative to connecting ring 705. The tabs 758 can also move slightly proximally until the tabs 758 confront lip 723.

After the tabs 758 pass through cutouts 717 and into channel 717a, either or both of the valve retainer 750 and/or connecting ring 705 are then rotated to move tabs 758 along or within the channel 711a, along internal surface 711. This brings tabs 758 out of alignment with the cutouts 717 and brings the valve retainer 750 and the connecting ring 705 into the locked position. In one example, the rotation can be approximately 90 degrees such that the tabs 758 are 90 degrees out of alignment with the cutouts 717. In the locked position, the lip 715 prevents removal of the tabs 758 from the channel 711a in the distal direction (while contact between the tabs 758 and the lip 723 prevents movement of the tabs 758 in the proximal direction relative to the valve retainer 750) without an appropriate counter rotation to once again align tabs 758 and cutouts 717. In this locked position, the valve retainer 750 and/or connecting ring 705 are not free to (i.e., cannot) move axially relative to one another and, in particular, the valve retainer 750 cannot move axially in a distal direction relative to the connecting ring 705.

As shown in FIG. 7C, the connecting ring 705 can be coupled or engaged with a first catheter 790 (e.g., via adhesives, welding (e.g., laser welded), or the like) and the valve retainer 750 can be coupled or engaged with a second catheter 795. In one example, the valve retainer 750 is screwed into the second catheter (e.g., steering catheter) and secured against rotation using adhesive. In other examples, the valve retainer 750 can be laser welded directly to the second catheter (e.g., steering catheter). The second catheter 795 can be concentrically nested within the first catheter 790. As shown, the second catheter 795 can have an internal threading 795a that can engage with external threading 766 of valve retainer 750. This can occur while the connecting ring 705 is in either the unlocked or locked position. Although not depicted, the external threading of external surface 713 can be engaged with a valve cover (such as valve cover 550a or valve cover 550b).

In one example, the first catheter 790 can be an outer sheath (such as outer sheath 405a) and the second catheter 795 can be a steering catheter (e.g., steering catheter 410a) or extension catheter (e.g., extension catheter 415a). In another example, the first catheter 790 can be an outer catheter (such as outer catheter 405b) and the second catheter 795 can be a steering catheter (e.g., steering catheter 410b) or intermediate catheter (e.g., intermediate catheter 415b).

In the unlocked state, the first catheter 790 is free to move axially in a distal direction relative to the second catheter 795 since the tabs 758 are in alignment with the cutouts 717. In the locked state, the first catheter 790 and the second catheter 795 are not free to (i.e., cannot) move axially relative to one another and, in particular, the outer catheter 790 cannot move axially in a distal direction relative to the inner catheter 795.

In the locked state, the valve cover, which is engaged with the external threading 713 of valve retainer 750, is axially locked to the first catheter 790. In other examples, the valve cover may be welded directly to the valve retainer 750. In this regard, the first catheter 790 can accommodate longitudinal compression forces exerted on the catheter assembly during delivery through the vasculature and prevent premature expression of the prosthetic heart valve. In other words, even if longitudinal compression forces are placed on a coiled or braided section of the first catheter 790, the first catheter 790 is axially locked to the second catheter 795 (because the connecting ring 705, which is fixed to the first catheter 790, is axially locked to the valve retainer 750, which in turn is fixed to the second catheter 795). Thus, because the second catheter 795 typically does not include a coiled and/or braided section similar to the first catheter 790, the second catheter 795 is not particularly susceptible to longitudinal compression. And since the second catheter 795 has significantly more resistance to longitudinal compression, the second catheter 795 provides resistance to longitudinal compression to the first catheter 790 when the first and second catheters are axially locked together.

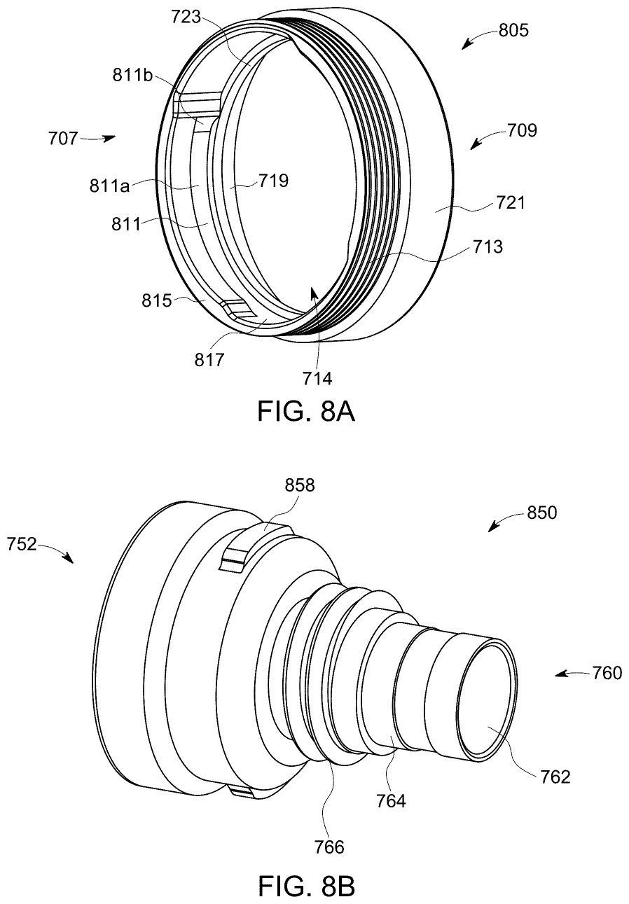

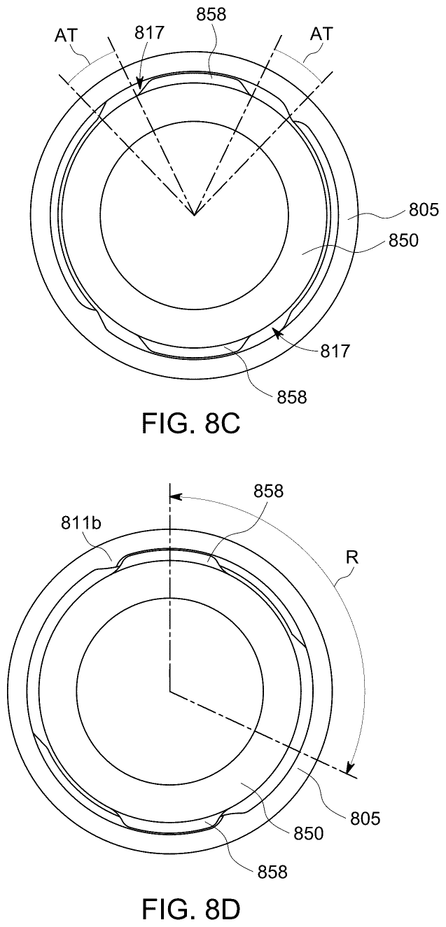

FIG. 8A is a perspective view of a connecting ring 805 and FIG. 8B is a perspective view of a valve retainer 850 according to one or more aspects of the disclosure. FIGS. 8C-D are transverse cross-sectional views of the connecting ring 805 of FIG. 8A coupled with the valve retainer 850 of FIG. 8B.

The connecting ring 805 can be similar in structure and function to the connecting ring 705, except the cutouts 817 formed in lip 815 can define a larger arc length as compared to the cutouts 717. Further, the internal surface 811 of connecting ring 805 can have a twist stop 811b formed at the end of channel 811a and between lips 815 and 723. The twist stop 811b can be a portion of the internal surface 811 that extends radially inward to prevent further rotation or over-rotation of the valve retainer 850 (and particularly of the tabs 858 of the valve retainer) relative to the connecting ring 805. The tabs 858 of valve retainer 850 can define a larger arc length relative to tabs 758 and the tabs 758 can extend further radially outward than the tabs 858. It should be understood that, in FIGS. 8A-B, components labeled in the 700 series of numerals are intended to be similar or identical to the similarly numbered parts shown and described in connection with FIGS. 7A-B.

As shown in FIG. 8C, the connecting ring 805 and valve retainer 850 are in an unlocked state. In this regard, the tabs 858 of the valve retainer 850 are received within the cutouts 817 but not yet rotated. There may be an angular tolerance AT on each side of the tabs 858 relative to the cutouts, allowing for the tabs 858 to be reliably received by the cutouts. The angular tolerance AT may be any suitable angle, and in one particular example the angle may be an approximately +/−15 to 20 degree (including approximately 18 degrees) tolerance on each side of the tabs 858 relative to the cutouts 817. As shown in FIG. 8D, the connecting ring 805 and valve retainer 850 are in a locked state, in which the tabs 858 have been rotated (R) approximately 115 degrees within the channel 811a of connecting ring 805. After the 115 degrees of rotation, the tabs 858 will confront the twist stop 811b, preventing further rotation. Meanwhile, the tabs 858 are unaligned with the cutouts 817, preventing the disengagement of the connecting ring 805 and valve retainer 850 and preventing the axial movement of connecting ring 805 relative to valve retainer 850.

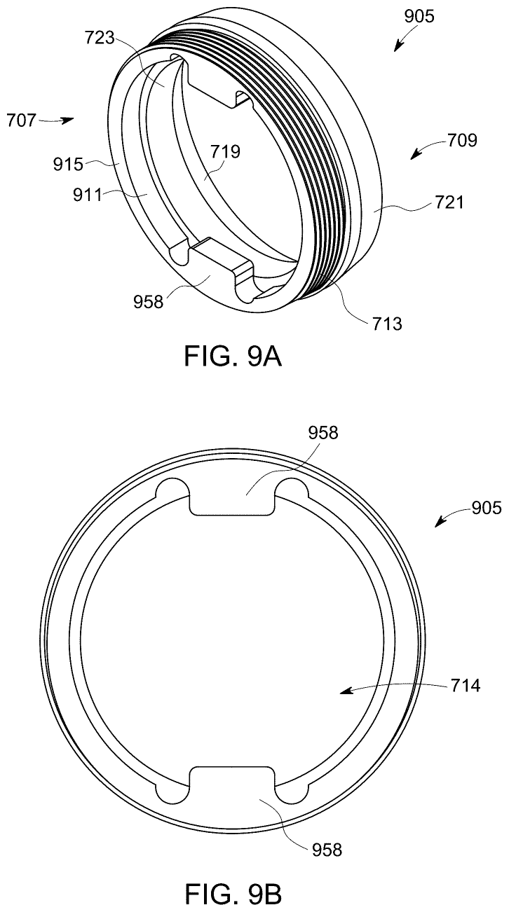

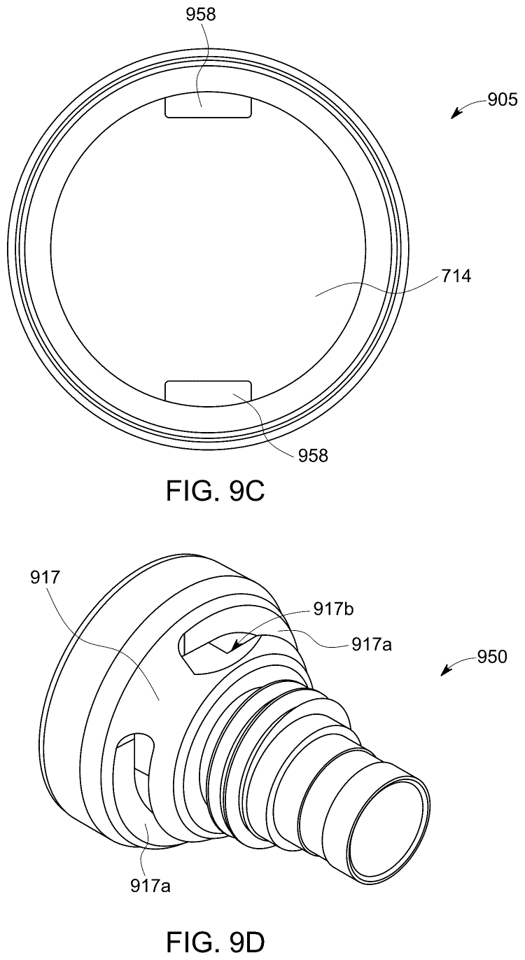

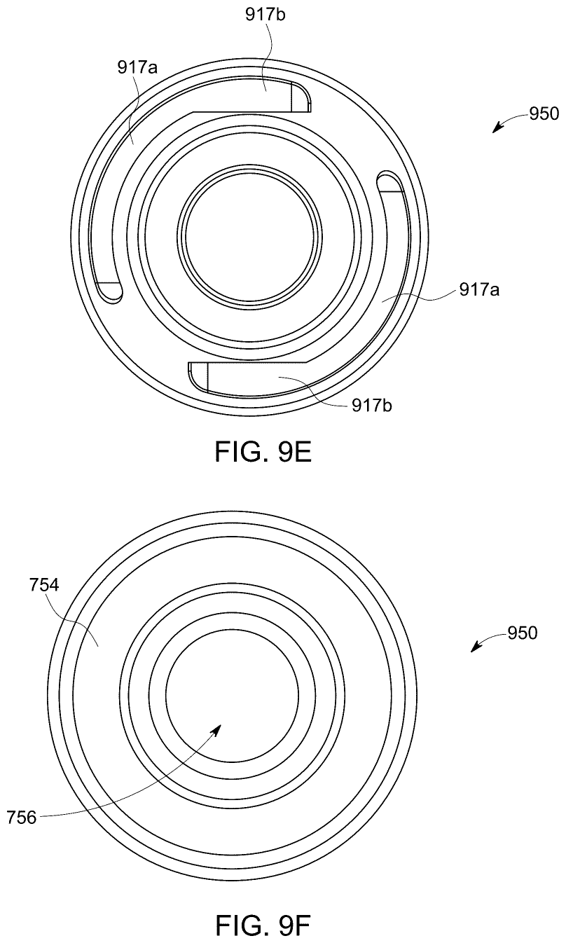

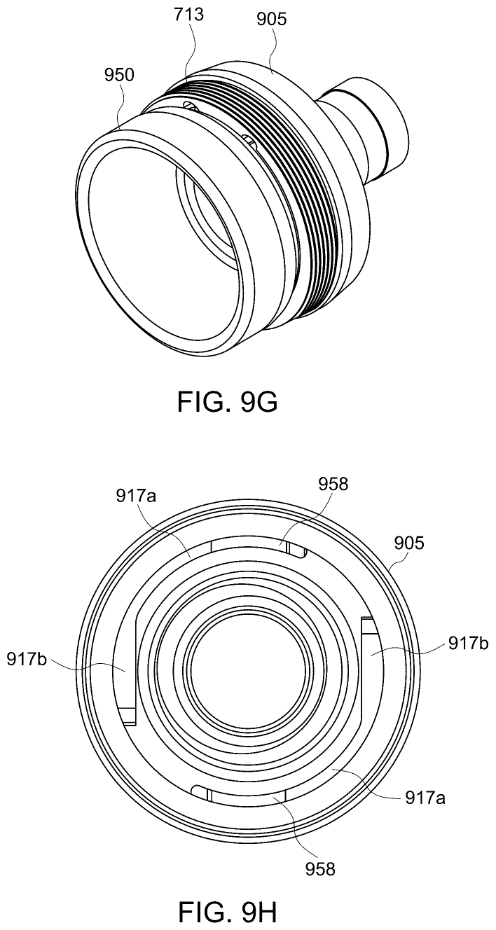

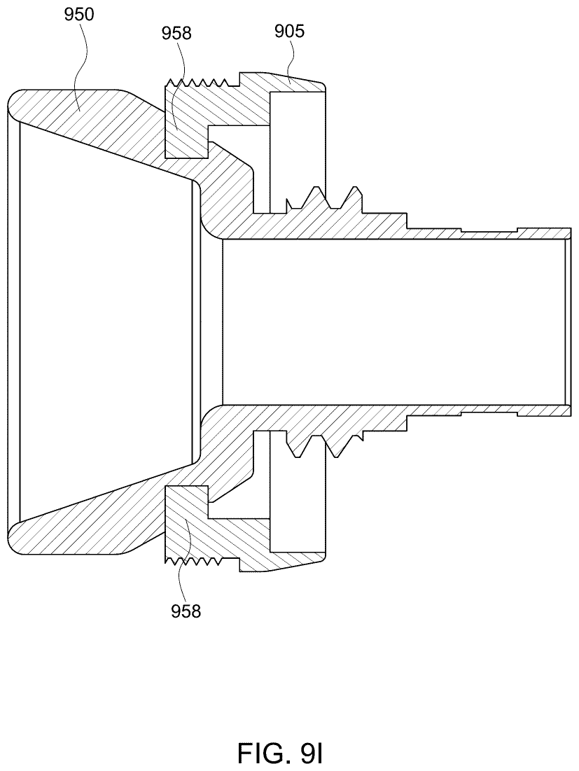

FIG. 9A-C are a perspective view, front view, and rear view of a connecting ring 905 according to one or more aspects of the disclosure.

The connecting ring 905 can be similar in structure and function to the connecting ring 705, except instead of a channel formed at internal surface 911, the connecting ring 905 can define a plurality of tabs 958 that extend radially inward relative to lip 915. It should be understood that, in FIGS. 9A-C, components labeled in the 700 series of numerals are intended to be similar or identical to the similarly numbered parts shown and described in connection with FIG. 7A.

FIGS. 9D-F are a perspective view, front view, and rear view of a valve retainer 950 according to one or more aspects of the disclosure. It should be understood that, in FIGS. 9D-F, components labeled in the 700 series of numerals are intended to be similar or identical to the similarly numbered parts shown and described in connection with FIG. 7B.

Corresponding to the tabs 958, the valve retainer 950 can define one or more channels 917a on an exterior surface 917. The channel 917a can define a plurality of cutouts 917b that can receive the tabs 958. The connecting ring 905 and the valve retainer 950 can be aligned such that the tabs 958 are aligned with the cutouts 917b. Once the tabs 958 are received within the cutouts 917b, the connecting ring 905 and/or the valve retainer 950 can be rotated such that the tabs 958 rotate within the channel 917a and are unaligned with the cutouts 917b, thereby preventing disengagement of the connecting ring 905 and valve retainer 950. It should be understood that, while connecting rings 705 and 805 effectively include recesses or female features (e.g., cutouts 717, 817) that receive the protrusions or male features (e.g., tabs 758, 858), the general configuration of connecting ring 905 and valve retainer 950 is reversed in the sense that the connecting ring 905 includes the protrusions or male features while the valve retainer 950 includes the recesses or female features.

FIGS. 9G-I are a perspective view, transverse cross-sectional view, and longitudinal cross-sectional view, respectively, of the connecting ring 905 of FIGS. 9A-C coupled with the valve retainer 950 of FIGS. 9D-F according to one or more aspects of the disclosure. As shown, the tabs 958 are received within the channel 917a and the connecting ring 905 and valve retainer 950 are axially locked together, thereby preventing axial movement in a distal direction of the valve retainer 950 relative to the connecting ring.

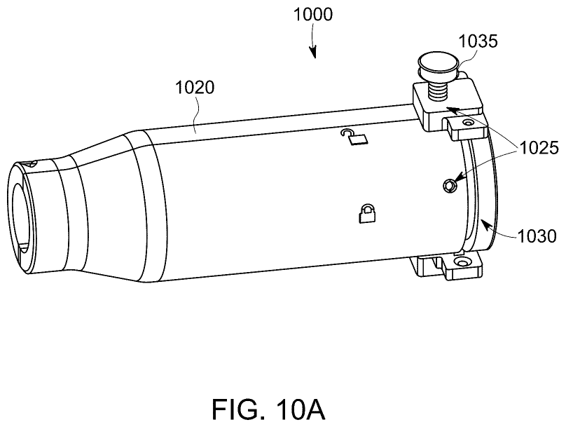

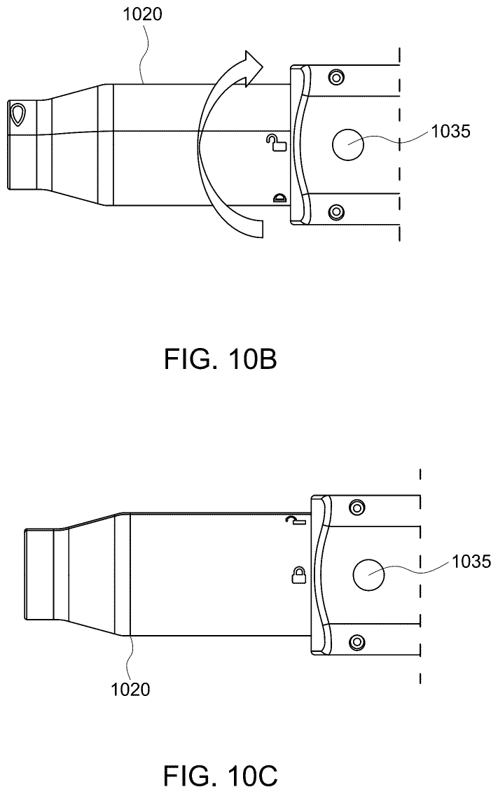

FIG. 10A is a perspective view of a locking assembly or cone assembly 1020 of a handle assembly 1000 operable with the delivery system of FIG. 1 (e.g., in place of locking assembly or cone assembly 120). The cone assembly 1020 can be rotated about its longitudinal axis in order to axially lock or unlock a first catheter (e.g., first catheter 790 which may be outer sheath 405a or 405b) relative to a second catheter (e.g., second catheter 795 which may be a steering catheter like steering catheter 410a, 410b, an extension catheter like extension catheter 415a, or an intermediate catheter like intermediate catheter 415b).

The cone assembly 1020 can include a plurality of positioning holes 1025, and a groove 1030. One of the positioning holes 1025 can correspond to a locked state and the other of the positioning holes 1025 can correspond to an unlocked state. In this regard, the cone assembly 1020 can be rotated relative to the housing of the handle assembly. In one example, the rotation can be approximately 90 degrees. This rotation can be guided by groove 1030, which can receive a portion of the housing of the handle assembly, ensuring that cone assembly 1020 is axially fixed relative to the handle assembly.

FIGS. 10B-C are side views of the cone assembly 1020 of FIG. 10A in the unlocked and locked state, respectively. As shown in FIGS. 10A-B, a screw 1035 of the handle assembly is received within the positioning hole 1025, thereby securing the cone assembly 1020 in the unlocked state. To move to the locked state, the screw 1035 is rotated to disengage from the positioning hole 1025 and the cone assembly 1020 is rotated. In other examples, the screw can be replaced with a spring-loaded pin configured to be snap into the positions holes 1025 when released and to be pulled outwardly to disengage. The cone assembly 1020 and/or handle assembly can optionally include stopping elements to prevent over rotation. In one example, the cone assembly 1020 is rotated until the locked state is reached. Upon reaching the locked state, the screw 1035 can be rotated to engage with the next positioning hole 1025, thereby securing the cone assembly in the locked state relative to the handle assembly. As shown in FIG. 1, the cone assembly 1020 can be operatively coupled to the catheter assembly such that rotation of the cone assembly 1020 can cause a corresponding rotation of the outer catheter (e.g., first catheter 790 or outer sheath 405a or 405b), allowing for the cone assembly 1020 to actuate between the locked and unlocked of the catheter assembly. In this regard, rotation of the cone assembly 1020 and outer catheter can cause relative rotation of the tabs and/or channels of the connecting ring and/or valve retainer, thereby providing the necessary axial alignment and/or unalignment.

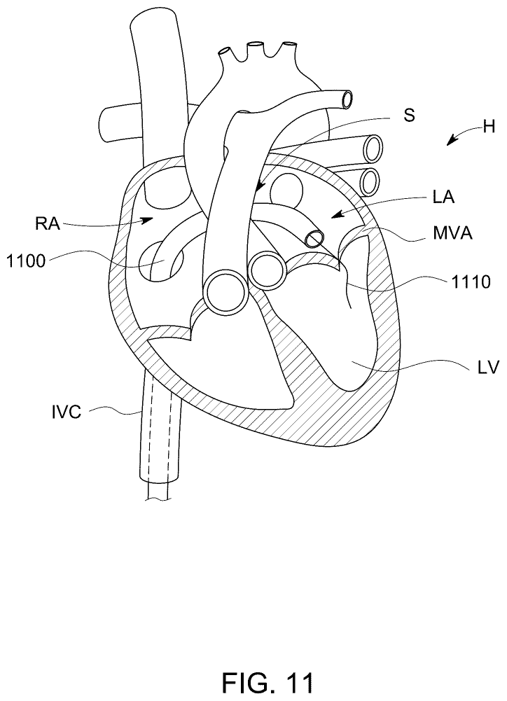

FIG. 11 is a schematic cutaway representation of a patient's heart H and a delivery route that may be followed by catheter assembly 1100 to reach the native mitral valve annulus MVA. It should be understood that catheter assembly 1100 may be representative of any of the catheter assemblies previously described herein. Using a transfemoral approach, catheter assembly 1100 may be inserted over guidewire 1110 into the patient's femoral vein and advanced through the inferior vena cava IVC to the right atrium RA. In a subsequent transseptal procedure, catheter assembly 1100 is advanced through a puncture in intra-atrial septum S into the left atrium LA. Once the catheter assembly 1100 is positioned at the desired location relative to the mitral valve annulus MVA, the catheter may be operated (e.g., by rotating valve cover retraction knob 115 to withdraw the valve cover 160 relative to the prosthetic heart valve maintained in a collapsed condition within the valve cover) to deploy a prosthetic mitral valve into the mitral valve annulus MVA.

In other implementations, such as for procedures associated with a tricuspid valve, catheter assembly 1100 may be advanced through the inferior vena cava IVC and into the right atrium RA, where it may then be positioned and used to perform the procedure related to the tricuspid valve. While many of the examples described herein relate to delivery to the native mitral valve annulus, one or more embodiments may be utilized in other cardiac procedures, including those involving the tricuspid valve or other cardiac valves.

Although one preferred method for accessing a targeted cardiac valve annulus is a transfemoral approach, it will be understood that the embodiments described herein may also be utilized where alternative approaches are used. For example, embodiments described herein may be utilized in a transjugular approach, transapical approach, transradial approach or other suitable approaches to the targeted anatomy. For procedures related to the mitral valve or tricuspid valve, the delivery of the prosthetic heart valve or other medical device is preferably carried out from an atrial aspect (i.e., with the distal end of catheter assembly 1100 positioned within the atrium superior to the targeted valve). The illustrated embodiments are shown from such an atrial aspect. However, it will be understood that the delivery of the medical devices described herein may also be carried out from a ventricular aspect.

Additional details regarding delivery systems and devices that may be utilized in conjunction with the components and features described herein are described in U.S. Patent Publication Nos. 2018/0028177A1, 2018/0092744A1, and 2020/0155804, the disclosures of which are hereby incorporated by reference herein.

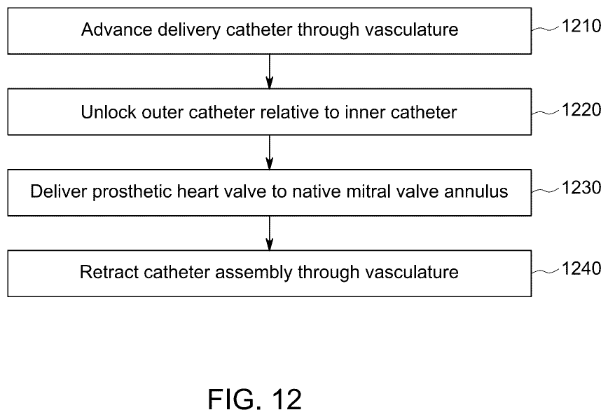

FIG. 12 is a flow chart depicting a method of operating a handle assembly during delivery of the prosthetic heart valve to the mitral valve annulus.

At block 1210, the delivery catheter is advanced through the vasculature. For example, the catheter assembly may be inserted into the patient's femoral vein and advanced until it reaches an opening of the inferior vena cava leading to the right atrium. The forces can be relatively high during insertion through the femoral vein until the valve cover reaches the inferior vena cava, where the larger diameter of the inferior vena cava reduces or eliminates frictional forces on the valve cover. While this advancing is occurring, a prosthetic heart valve (e.g., a self-expanding prosthetic mitral valve) may be maintained in a collapsed condition within a valve cover of the catheter assembly. Importantly, in some embodiments, an introducer sheath or introducer device may be omitted from the procedure such that the catheter assembly is inserted directly into the patient's femoral vein without a separate introducer catheter within the femoral vein through which the catheter assembly is advanced. During block 1210, the catheter assembly can be in the locked state and thus the valve cover and outer sheath (e.g., first catheter 790) can be axially locked relative to an inner catheter (e.g., second catheter 795, which may be one of the steering catheters described above, or the extension or intermediate catheter described above). In the locked state, force can be applied to the catheter assembly to push the assembly through the vasculature without the risk of premature unsheathing and/or expression of the prosthetic heart valve. In this locked state, the second catheter can be an extension or steering catheter, which can accommodate the compression forces exerted during delivery, and thus provide stability to the first catheter (or outer sheath) to prevent collapsing of any coiled or braided sections of the first catheter.

At block 1220, second catheter can be unlocked relative to the first catheter (e.g. by unlocking the connection ring relative to the valve retainer). This can be done by rotating the cone assembly, which can disengage the respective tabs and/or channels of the connector ring and/or valve retainer, thereby allowing for axial movement of the valve retainer relative to the connecting ring. The unlocking can occur at any time during advancement of the catheter assembly, and in one example occurs at or near the opening of the inferior vena cava leading to the right atrium. In another example, the unlocking can occur when the valve cover is in the vena cava and is in a relatively straight (unbent or undeflected) position, as the rotational forces for unlocking are minimized in the straight position as compared to a bent or deflected position. It may be desirable to keep the catheter assembly in the locked condition while the catheter assembly is in the femoral vein to avoid the premature valve deployment described above. However, while the catheter assembly is in the locked condition and the first catheter is locked to the second catheter, it may be difficult to bend the catheter to any significant degree which may be required to guide the catheter through the heart. In many patient anatomies, the femoral vein has a relatively non-tortuous path between a typical entry point in the groin to the point at which the inferior vena cava IVC opens into the right atrium RA. Thus, the more difficult bending operation of the catheter assembly may not be necessary prior to the catheter assembly exiting the inferior vena cava IVC. But upon exiting the inferior vena cava IVC, the bending or steering operation may be highly desirable and thus unlocking the catheter assembly at or near the inferior vena cava IVC is one suitable option.

At block 1230, with the valve retainer unlocked relative to the connecting ring, the prosthetic heart valve can be delivered to the native mitral valve annulus, for example through the right atrium RA, septum S and left atrium LA. Various steering or bending operations may be performed to guide the steering catheter along this path and to eventually position the valve cover near or in the mitral valve annulus MVA. When the valve cover is in the final desired position, the prosthetic heart valve may be deployed by retracting the valve cover relative to the catheter to which the valve retainer is connected. Since the valve retainer is in the unlocked state at this point, the valve cover is free to retract axially relative to the valve retainer, and thus the prosthetic heart valve is free to express from the valve cover of the catheter assembly and into the native mitral valve annulus.

At block 1240, the catheter assembly is retracted through the vasculature and removed from the patient. During removal, the catheter assembly can be in the locked or unlocked state, although it may be preferable to keep the catheter assembly in the unlocked state for easier bending if needed.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A delivery system for delivering a prosthetic heart valve, the delivery system comprising:
a handle assembly;
a catheter assembly extending from the handle assembly, the catheter assembly having a first catheter and a second catheter positioned radially inside the first catheter;
a connecting ring fixed to a distal end portion of the first catheter; and
a valve retainer fixed to a distal end portion of the second catheter, the valve retainer configured to receive at least a portion of the prosthetic heart valve therein,
wherein the catheter assembly has i) a locked state in which the valve retainer is axially locked relative to the connecting ring, and ii) an unlocked state in which the valve retainer is axially movable relative to the connecting ring, the handle assembly being configured to actuate the catheter assembly between the locked state and the unlocked state.

2. The delivery system of claim 1, wherein the first catheter has a coiled or braided section positioned proximal to the connecting ring.

3. The delivery system of claim 2, wherein the second catheter has a greater resistance to longitudinal compression compared to the first catheter.

4. The delivery system of claim 3, wherein:
the first catheter is an outer sheath of the catheter assembly; and
the second catheter is a steering catheter or an extension catheter.

5. The delivery system of claim 3, wherein:
the first catheter is an outer sheath of the catheter assembly; and
the second catheter a steering catheter or an intermediate catheter.

6. The delivery system of claim 3, wherein the connecting ring defines a slot configured to receive a tab of the valve retainer.

7. The delivery system of claim 3, wherein the valve retainer defines a slot configured to receive a tab of the connecting ring.

8. The delivery system of claim 3, wherein the handle assembly includes a locking assembly such that rotation of the locking assembly relative to other components of the handle assembly actuates the catheter assembly between the locked state and the unlocked state.

9. The delivery system of claim 1, wherein the catheter assembly includes a valve cover fixed to the connecting ring, the valve cover configured to maintain at least part of the prosthetic heart valve in a collapsed condition.

10. The delivery system of claim 9, wherein in the locked state of the catheter assembly, the valve cover is axially locked relative to the valve retainer, thereby preventing premature expression of the prosthetic heart valve from the valve cover.

11. The delivery system of claim 10, wherein in the unlocked state of the catheter assembly, the valve cover is configured to retract proximally along with the first catheter relative to the second catheter and relative to the valve retainer, thereby allowing the for expression of the prosthetic heart valve from the valve cover.

12. The delivery system of claim 9, wherein the valve cover is threadedly coupled to the connecting ring.

13. The delivery system of claim 9, wherein the wherein the handle assembly includes (i) a valve cover retraction knob and (ii) a locking assembly, such that rotation of the valve cover retraction knob in the unlocked state of the catheter assembly proximally withdraws the valve cover relative to the valve retainer, and rotation of the locking assembly relative to other components of the handle assembly actuates the catheter assembly between the locked state and the unlocked state, the valve cover retraction knob being positioned distal to the locking assembly.

14. The delivery system of claim 1, wherein the valve retainer is threadedly coupled to the second catheter.

15. A method of delivering a prosthetic heart valve, comprising:
advancing a catheter assembly through a vasculature while the catheter assembly is in a locked state in which a first catheter is axially locked relative to a second catheter nested within the first catheter, and while the prosthetic heart valve is maintained in a collapsed condition within a valve cover coupled to a distal end of the first catheter;
actuating the catheter assembly to an unlocked state, thereby allowing for axial movement of the first catheter relative to the second catheter; and
retracting the valve cover and the first catheter relative to the second catheter to deploy the prosthetic heart valve into a native mitral valve annulus.

16. The method of claim 15, wherein actuating the catheter assembly to the unlocked state is performed when a distal end of the catheter assembly is positioned within or adjacent to an opening of an inferior vena cava leading to a right atrium.

17. The method of claim 15, wherein actuating the catheter assembly to the unlocked state includes rotating a locking assembly of a handle assembly, the catheter assembly extending from the handle assembly.

18. The method of claim 15, wherein the catheter assembly includes a connecting ring that couples the valve cover to the distal end of the first catheter, and a valve retainer coupled to a distal end of the second catheter.

19. The method of claim 18, wherein actuating the catheter assembly to the unlocked state includes rotating the first catheter relative to the second catheter such that the valve retainer rotates relative to the connecting ring.
